(19) United States
(12) Patent Application Publication
(10) Pub. No.: US 2007/0009984 A1
(43) Pub. Date: Jan. 11, 2007

(54) Churning methods for separating microorganisms from a food matrix for biodetection

(76) Inventors: Amanda Votaw, Cody, WY (US); Paul E. Johnson, Laramie, WY (US)

Correspondence Address: JENNIFER L. BALES, MOUNTAIN VIEW PLAZA, 1520 EUCLID CIRCLE, LAFAYETTE, CO, 80026-1250, US

(21) Appl. No.: 11/455,232

(22) Filed: Jun. 16, 2006

Related U.S. Application Data
(63) Continuation-in-part of application No. 10/382,253, filed on Mar. 5, 2003, now abandoned.

(51) Int. Cl. C12Q 1/04

(52) U.S. Cl. 435/34

(57) ABSTRACT

A churning process with several variations that removes bacteria from food by mechanical means while adequately filtering the food matrix sufficiently for analysis in cytometer. Churning stirs, agitates, shakes, shears or compresses the food sufficiently to dislodge bacteria from food particles, without chopping, crushing, or cutting the food. In a first variation, the specimen (e.g. ground beef) is combined with water or liquid buffer, stomached, and the liquid collected for measurement in a flow cytometer. In a second variation, the specimen is combined with water or liquid buffer, vortexed and the liquid supernatant is collected for measurement in a flow cytometer. In a third variation, the specimen is combined with water or liquid buffer, placed in a sonicating water bath and the supernatant collected for measurement in a flow cytometer.

### BACKGROUND OF THE INVENTION

This application claims the benefit of U.S. Provisional Application No. 60/361,994, filed Mar. 5, 2002 and is a continuation in part of U.S. patent application Ser. No. 10/382,253, filed Mar. 5, 2003.

### FIELD OF THE INVENTION

Some of the recently published techniques for removing bacteria from ground beef include:

1. Using a combination of detergent and enzyme treatment with differential centrifugation prior to detection by plate count and DEFT (Direct Epifluorescent Filter Technique) (Rodrigues-Szulc, U. M. et al., 1996).

2. Using surface adhesion onto polycarbonate filters (Sheridan et al., 1998).

3. Chopping and stirring the food with a bladed blender and subsequently centrifuging the food to separate fat, aqueous, and tissue layers, and the subsequent removal of the aqueous layer which presumably contains the great majority of bacteria (Carroll et al., 2000).

These techniques are all flawed and do not produce the required separation of bacteria from food matrix.

In particular, it has been discovered that methods that chop, cut up, or crush the food do not produce good or consistent results. For example, attempts to replicate the Carroll et al. experiment above counted only between 1% and 17% of the bacteria in the food, so results were both poor and inconsistent. In part this is because the bacteria adhere to the crushing or chopping surface. In addition, centrifugation by itself did not separate the bacteria from the layers of tissue and fat. Bacteria were found in the tissue layer, the fat layer, the aqueous layer and on the blender blades.

“Many rapid methods have been developed that are capable of detecting low numbers of bacteria in pure culture, but these do not always work efficiently when applied to complex food materials, owing to the presence of particulate and soluble components which cause background interference” (Rodrigues-Szulc, U. M. et al., 1996). In order to sensitively detect pathogenic bacteria in food to remove bacteria from the food and concentrate it prior to testing, new testing procedures need to be developed (ibid.).

One of the chief means of separating microorganisms from food uses an initial “stomaching” which homogenizes the food to first order (Sharpe and Jackson, 1972). However, “the method only achieves partial success in removing micro-organisms as it fails to disrupt the many physicochemical forces involved in the adhesion of bacteria to food surfaces. If the target organisms remain attached to very small particles after the initial stomaching stage, the effectiveness of subsequent separation processes will be severely impaired.” (Rodrigues-Szulc, U. M. et al., 1996).

These examples from recent literature “teach against” using mechanical means, such as stomaching, to separate bacteria from food prior to testing.

A need remains in the art for new mechanical methods of separating bacteria from food and measuring the concentration of bacteria more accurately, by churning the food with a buffer and then filtering the churned mixture.

### REFERENCES

Carroll S. A., L. E. Carr, E. T., Mallinson, C. Lamichanne, B. E. Rice, D. M. Rollins, and Joseph, S. W. 2000. “Development and Evaluation of a 24-hour Method for the Detection and Quantification of Listeria monocytogenes in Meat Products.” J. Food Prot., 63, p. 347-353.
Rodrigues-Szulc, U. M., Ventoura, G., Mackey, B. M., and Payne, M. J. 1996. “Rapid Physicochemical Detachment, Separation and Concentration of Bacteria from Beef Surfaces.” J. Applied Bacteriology, 80, p. 673-681.
Sharpe, A. N. and Jackson, A. K. 1972. “Stomaching: a New Concept in Bacteriological Sample Preparation.” Applied Microbiology, 24, p. 175-178.
Sheridan, J. J., Logue, C. M., McDowell, D. A., Blair, I. S., Hegarty, T., and Toivanen, P. 1998. “The Use of a Surface Adhesion Immunofluorescent (SAIF) Method for the Rapid Detection of Yersinia enterocolitica Serotype O:3 in Meat,” J. Applied Microbiology, 85, p. 737-745.

### SUMMARY OF THE INVENTION

The present invention comprises new mechanical methods of separating bacteria from food and measuring the concentration of bacteria more accurately, by churning the food with a buffer and then filtering the churned mixture. The methods of the present invention are “churning” methods, wherein churning is defined as a process which stirs, agitates, shakes, shears or compresses the food sufficiently to dislodge bacteria from food particles, without chopping, crushing, or cutting the food.

Three variations on the churning process of the present invention for separating microorganisms from a food matrix for biodetection are as follows:

1. The specimen (e.g. ground beef) is combined with a fluid such as water or liquid buffer to form a sample, the sample is stomached, and the resulting liquid is collected for measurement in a flow cytometer. Stomaching is a process wherein the food specimen and the liquid are kneaded together in a divided bag having a filter layer separating the first bag portion containing the specimen from the second bag portion. As the food and liquid are kneaded, the liquid passes through the filter layer into the second bag portion. The liquid in the second portion after the stomaching process contains the majority of the bacteria from the sample, and the percentage of the bacteria extracted is consistent, so long as a consistent mechanical stomacher is used.

2. The specimen is combined with a fluid such as water or liquid buffer to form a sample, the sample is vortexed and the liquid supernatant is collected for measurement in a flow cytometer. Vortexing revolves the specimen and liquid around an axis rapidly, so that the combination is swirled in its container. The food specimen is stirred and sheared by this vortexing process, and this causes the bacteria to separate from the food and enter the liquid. Again the concentration of bacteria in the liquid is measured.

3. The specimen is combined with a fluid such as water or liquid buffer to form a sample, the sample is placed in a container such as a test tube and sonicated in a sonicating water bath, and the supernatant collected for measurement in a flow cytometer. Sonicating vigorously shakes or agitates the food particles, separating the bacteria from the food. Again the bacteria enters the liquid and the concentrion of bacteria in the liquid is measured.

Each method generally includes a premixing step, wherein food is mixed with the buffer fluid to form a slurry, prior to the churning step. For example, ground beef might be mixed with water for about a minute with a lab spatula to form a slurry, and this slurry would then be placed in the stomacher or other churning device. In this example, the residue on the lab spatula was washed into the slurry with a bit of buffer. Each method includes a filtering step to separate the liquid and the bacteria it now contains from the remaining food specimen prior to measuring the concentration of bacteria in the liquid in a cytometer or the like. The resulting liquid may be further filtered if desired.

### BRIEF DESCRIPTION OF THE DRAWINGS

FIG. 1 shows a flow diagram illustrating a churning first process of separating microorganisms from a food matrix for biodetection according to the present invention, utilizing stomaching.

FIG. 2 shows a flow diagram illustrating a second churning process of separating microorganisms from a food matrix for biodetection according to the present invention, utilizing vortexing.

FIG. 3 shows a flow diagram illustrating a third churning process of separating microorganisms from a food matrix for biodetection according to the present invention, utilizing a sonicating water bath.

### DETAILED DESCRIPTION OF THE PREFERRED EMBODIMENTS

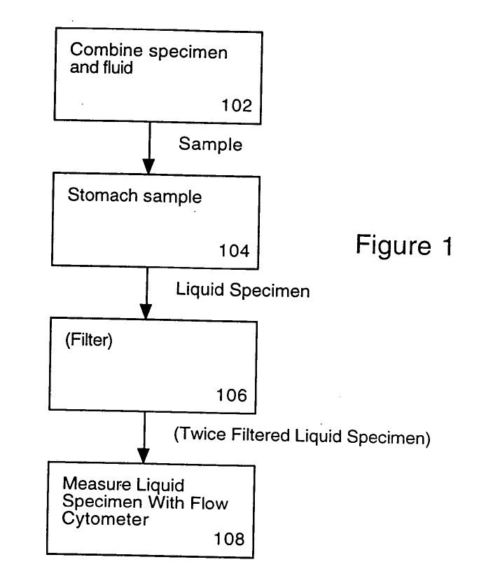

FIGS. 1-3 illustrate three variations on the churning process of the present invention. Each method efficiently, quickly, and inexpensively removes bacteria from food by mechanical means while adequately filtering the liquid from the food matrix sufficiently for analyzing the bacteria levels in the liquid in a cytometer, preferably a wide-flow-cross-section flow, flow cytometer. Each process performs the separation efficiently, especially with ground beef (which was tested with E. coli K-12):

FIG. 1 illustrates a process using stomaching. In step 102, the specimen (e.g. ground beef) is combined with water or liquid buffer to form a sample. In step 104, the specimen is manually stomached in a bag (preferably using an automatic stomacher for most consistent results), which mixes the sample and extracts the liquid including the bacteria, while leaving large food particles behind. A second filtering step 106 may be performed after stomaching. In step 108, the liquid specimen is measured in a cytometer.

In a preferred embodiment, VWR-brand Filtra-bag stomacher bags are used during stomaching step 104. These contain a 310-micron inner filter allowing the collection of liquid without the contamination of particles larger than 310 microns. The sample is placed in one side of the stomacher bag, and the slurry is kneaded manually or by a stomacher device. The liquid flows through the filter layer in the stomacher bag to the other side of the bag. This liquid can then be vacuum filtered through a 105-micron polystyrene filter (implementing optional step 106). The filtrate is flowed through a flow-cytometer flow-cell with a large (around 2 mm) cross-section while maintaining bacterial integrity.

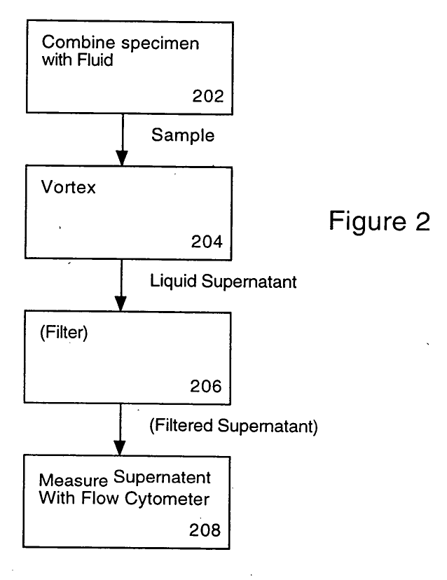

FIG. 2 illustrates a process using vortexing. In step 202, the specimen (e.g. ground beef) is combined with water or liquid buffer to form a sample. In step 204, the sample is vortexed, resulting in a supernatant including bacteria. A filtering step 206 is performed after vortexing to separate the supernatant from the remaining food specimen. In step 208, the supernatant is measured in a cytometer. In a preferred embodiment, the ground beef and fluid sample is placed in a 50 ml conical plastic tube (e.g. a tube normally used in centrifuges) with 44.75 ml buffer, vortexed at a 90° angle for 2 minutes at 2000 rpm and the liquid supernatant is collected for measurement in a flow cytometer. The vortexing step 204 vibrates the specimen in a circular pattern, generating a vortex.

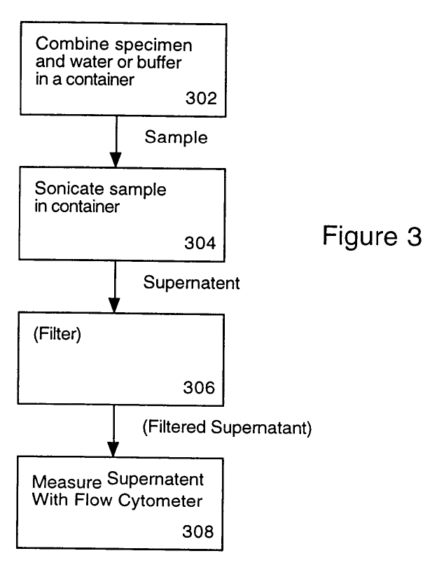

FIG. 3 illustrates a process using a sonicating water bath. In step 302, the specimen (e.g. ground beef) is combined with water or liquid buffer to form a sample. In step 304, the sample is placed in a container such as a test tube and sonicated in a sonicating water bath, resulting in a supernatant including bacteria. A filtering step 306 is performed next. In step 308, the supernatant is measured in a cytometer. In a preferred embodiment, three grams of ground beef is placed in 26.85 ml buffer for 30 minutes in a sonicating water bath and the supernatant collected for measurement in a flow cytometer.

For all processes, a preferred flow cytometer device (not shown) is a large diameter (around 2 mm cross section) flow, imaged transverse to the flow with a CCD camera.

Results of all three churning methods show high-efficiencies for manual mixing/stomaching and vortexing with lower efficiencies for sonication. Typical efficiencies were at the 70% level. (In other words, ca. 70% of the E. coli K12 in the beef specimen were recovered as determined by spread plate counting.)

What is claimed is:

1. A churning method of separating microorganisms from a food matrix specimen for biodetection according to the present invention, where churning is defined as stirring, agitating, shaking, shearing or compressing the food specimen sufficiently to dislodge bacteria from food particles, without chopping, crushing, or cutting the food, the method comprising the steps of:
combining the food specimen with a fluid to generate a sample;
churning the sample to dislodge the bacteria from the food specimen and cause it to suspend in the fluid;
filtering the sample to separate the fluid and the suspended bacteria from the remaining food specimen, forming a liquid specimen; and
measuring the concentration of bacteria in the liquid specimen in a cytometer.

2. The method of claim 1 wherein the churning method comprises stomaching.

3. The method of claim 2, wherein the stomaching step is performed in a stomacher bag having approximately a 310-micron inner filter layer.

4. The method of claim 1 wherein the churning method comprises vortexing.

5. The method of claim 4, wherein the vortexing step includes the steps of:
placing the sample into a conical plastic tube;
vortexing the sample at approximately a 90° angle.

6. The method of claim 4 wherein the vortexing step lasts approximately two minutes at approximately 2000 rpm.

7. The method of claim 1 wherein the churning method comprises sonicating.

8. The method of claim 7, wherein the combining step comprises placing approximately three grams of ground beef and approximately 26.85 ml buffer in a test tube and the sonicating step comprises sonicating for 30 minutes in a sonicating water bath.

9. The method of claim 1, further including the step of additionally filtering the liquid specimen prior to the measuring step.

10. The method of claim 9, wherein the additional liquid filtering step comprises vacuum filtering.

11. The method of claim 1, wherein the fluid is water.

12. The method of claim 1, wherein the fluid is buffer.

13. The method of claim 1, wherein the measuring step is performed using a flow cytometer.

14. The method of claim 13, wherein the flow-cell has approximately a 2 mm cross-section.
